(19) United States
(12) Patent Application Publication
(10) Pub. No.: US 2005/0260256 A1
(43) Pub. Date: Nov. 24, 2005

(54) Methods and apparatus for extrusion of vesicles at high pressure

(76) Inventors: Knut R. Hill, Ypsilanti, MI (US); Wendi Rodrigueza, Ann Arbor, MI (US); Gavin A. Sy, Ann Arbor, MI (US)

Correspondence Address: JONES DAY, 222 EAST 41ST ST, NEW YORK, NY, 10017, US

(21) Appl. No.: 10/256,767

(22) Filed: Sep. 27, 2002

Related U.S. Application Data
(60) Provisional application No. 60/326,032, filed on Sep. 28, 2001.

(51) Int. Cl. A61K 9/127; B01J 13/04; B01J 13/02

(52) U.S. Cl. 424/450; 264/4.1

(57) ABSTRACT

This invention relates in general to methods and devices for producing vesicles, including micelles, and particularly liposomes, by extruding solutions comprising materials capable of forming vesicles through a screen membrane at high pressure.

This application claims the benefit under 35 U.S.C. §119 of U.S. provisional application No. 60/326,032, filed Sep. 28, 2001, which is incorporated herein by reference in its entirety.

### FIELD OF THE INVENTION

This invention relates in general to methods and devices for producing vesicles, including micelles, and particularly liposomes, by extruding solutions comprising materials capable of forming vesicles through a screen membrane at high pressure.

### BACKGROUND

The use of liposomes for drug delivery has been proposed for a variety of drugs, particularly those which are administered parenterally. Liposomes have the potential for providing controlled “depot” release of the administered drug over an extended time period, and of reducing side effects of the drug, by limiting the concentration of free drug in the bloodstream. Liposomes also can alter the tissue distribution and uptake of drugs, in a therapeutically favorable way, and can increase the convenience of therapy, by allowing less frequent drug administration. These effects can be enhanced by attaching ligands that target the liposomes to certain types of cells or tissues within the body. Liposome drug delivery systems are reviewed in Poznansky, et al., 1984, Pharmacol. Rev. 36:277-336.

Generally, the optimal size of liposome for use in parenteral administration is between about 70 and 300, and up to about 400 nm in diameter. Liposomes in this size range can be sterilized by passage through conventional depth filters having particle size discrimination of about 200 nm. This size range of liposomes also favors biodistribution in certain target organs, such as liver, spleen, and bone marrow, and gives more uniform and predictable drug-release rates and stability in the bloodstream. Liposomes whose sizes are less than about 400 nm also show less tendency to aggregate on storage, and are thus generally safer and less toxic in parenteral use than larger-size liposomes.

A variety of techniques for preparing liposomes have been proposed, including, for example, sonication, extrusion, high pressure homogenization, microfluidization, detergent dialysis, calcium-induced fusion of small liposome vesicles, and ether-infusion methods. See, e.g., U.S. Pat. No. 4,186,183; U.S. Pat. No. 4,217,344; U.S. Pat. No. 4,261,975; U.S. Pat. No. 4,485,054; U.S. Pat. No. 4,774,085; U.S. Pat. No. 4,946,787; U.S. Pat. No. 6,139,871; PCT Publication No. WO 91/17424, Deamer, et al., 1976, Biochim. Biophys. Acta, 443:629-34; Fraley, et al., 1979, Proc. Natl. Acad. Sci. USA, 76:3348-52; Hope, et al., 1985, Biochim. Biophys. Acta, 812:55-65; Mayer, et al., 1986, Biochim. Biophys. Acta, 858:161-68; Williams, et al., 1988, Proc. Natl. Acad. Sci. USA, 85:242-46 and Szoka, et al., 1980, Biochim. Biophys. Acta, 601:559-71. Typically, these methods yield liposomes which are heterodisperse, and predominantly greater than about 1 micron in size. These initial heterodisperse suspensions can be reduced in size and size distribution by a number of known methods. One size-processing method which is suitable for large-scale production is homogenization. Here the initial heterodisperse liposome preparation is pumped under high pressure through a small orifice or reaction chamber. The suspension is usually cycled through the reaction chamber until a desired average size of liposome particles is achieved. A limitation of this method is that the liposome size distribution is typically quite broad and variable, particularly in the size range of 100 nm average liposome diameter, depending on a number of homogenization cycles, pressures, and internal temperature. Also, the processed fluid has the potential to pick up metal and oil contaminants from the homogenizer pump, and may be further contaminated by residual chemical agents used to sterilize the pump seals.

Sonication, or ultrasonic irradiation, is another method that is used for reducing liposome sizes. This technique is useful especially for preparing small unilameller vesicles (SUVs), in the 25 to 80 nm size range. However, a narrow size distribution of liposomes can only be achieved at liposome sizes of about 50 nm, i.e., after the liposomes have been reduced to their smallest sizes. These very small liposomes have limited drug carrying or loading capacity and less favorable biodistribution properties than those in the 100 to 400 nm size range, as noted below. The processing capacity of this method is also quite limited, since long-term sonication of relatively small volumes is required. Also, heat build-up during sonication can lead to peroxidative damage to lipids, and sonic probes shed titanium particles which are potentially quite toxic in vivo.

A third general size-processing method is based on liposome extrusion through a uniform pore-size membrane made of polycarbonate or another similar material. See, Szoka, et al., 1978, Proc. Natl. Acad. Sci. USA, 75:4194-8. This procedure has advantages over the above homogenization and sonication methods in that a variety of membrane pore sizes is available for producing liposomes in different selected size ranges, and in addition, the size distribution of the liposomes can be made quite narrow, particularly by cycling the material through the selected-size filter several times. A number of techniques for extruding liposomes have been reported. For example, U.S. Pat. No. 4,927,637 describes a method of extruding lipids through a tortuous-path nylon, TUFFRYN® (Pall Corp., East Hills N.Y.), polysulfone, polypropylene or scintered steel membrane at low pressure (e.g., 250 lbs/inch² (psi)). U.S. Pat. No. 5,008,050 teaches a method of extruding liposomes through a polycarbonate filter at between 100 and 700 psi or more. U.S. Pat. No. 4,737,323 teaches a method of producing liposomes by extruding a suspension of lipids through a ceramic membrane at 200 to 250 psi.

However, membrane extrusion methods have several drawbacks in large-scale processing. For one, the pores in the membrane tend to clog, particularly when processing concentrated suspensions and/or when the liposome sizes are substantially greater than the membrane pore sizes. Most production-scale extrusion devices do not allow for backflushing to clear the membranes. Replacing the clogged membrane with a fresh membrane opens the extrusion system to the environment and poses a risk of product contamination, even if the membranes are backflushed. The membranes cannot be steam-sterilized in place, with a high degree of confidence, due to their inherent fragility. Whatever method is employed to overcome a clogged or fouled membrane, it adds time and expense to the extrusion process.

The shortcomings of the currently available liposome extrusion methods are particularly acute when certain types of lipids are extruded. Lipid bilayers adopt a crystalline phase below temperature T_{c}′, a gel phase between temperatures T_{c}′ and T_{c}, and a liquid crystalline state above temperature T_{c}. See, Lasic, 1997, Liposomes in Gene Delivery, CRC Press LLC, Boca Raton 67-71. Lipids with T_{c} values greater than about room temperature can be especially difficult to extrude through polycarbonate membranes. The value of T_{c} for a particular lipid depends on a number of factors, including the length and degree of saturation of the lipid's hydrocarbon chains. Lipids with longer, more saturated hydrocarbon chains tend to have higher T_{c} values (and so tend to be more difficult to extrude through polycarbonate membranes) than lipids with shorter, less saturated hydrocarbon chains. Lipids can also be difficult to extrude because of impurities in the starting material, for example, contamination with resins that are a byproduct of the manufacturing process. Lipids that are difficult to extrude also have slower flow rates and foul or clog the membranes more readily than other lipids. As explained above, clogged or fouled membranes must be cleaned or replaced, increasing the time and cost of production and the possibility of contamination.

Thus, there is a need for methods and devices for extruding lipids, particularly difficult to extrude lipids, in a time- and cost-efficient manner, with a reduced incidence of membrane clogging or fouling, and with a reduced likelihood of contamination.

### SUMMARY OF THE INVENTION

This invention relates to methods and devices that are particularly useful for the manufacture of vesicles, including micelles, and particularly liposomes, and pharmaceutical products containing vesicles, micelles or liposomes. More specifically, we have discovered that extruding lipids through hydrophilic screen membrane(s) under high pressure results in greatly improved flow rates while still producing vesicles of the desired size and lamellarity. The screen membranes, as will be described in detail below, are membranes that have pores whose channels through the membrane on average exhibit essentially straight lines. The methods and devices of the present invention are particularly useful for producing vesicles of a desired size and lamellarity from lipids that are difficult to extrude using conventional techniques.

We have further discovered that extrusion of a lipid preparation through PORETICS™ membranes (Osmonics, Minnetonka Minn.) results in greatly increased flow rates compared to extrusion through other commercially available membranes.

We have further discovered that using a hydrophilic membrane in accordance with the invention and at high extrusion pressure results in a decrease of the size of the vesicles that are produced by the extrusion.

The methods and devices of the invention can be operated in a relatively problem-free manner, with reduced membrane clogging or fouling, at high throughput volumes, and in a large-scale operation. Thus, the methods and devices of the invention are well-suited for use in manufacturing liposomes.

In one aspect the invention provides a method of producing a suspension of liposomes comprising extruding an aqueous suspension of lipids through a hydrophilic membrane, particularly a screen membrane(s), at high pressure. The invention provides a method of producing a suspension of liposomes of a uniform size distribution. That is, the liposomes created by the method of the invention exhibit little variance in the average size distribution. In addition, the invention provides a method for producing liposomes of any form, for example, the liposome suspension can be lyophilized to produce a powder.

In another aspect the invention provides a method of producing a suspension of liposomes comprising extruding an aqueous suspension of lipids through an angled pore screen membrane at high pressure. Angled pore screen membranes, as will be described in detail below, are screen membranes wherein the angles formed by the pores relative to the plane of the face of the membrane is less than about 90°.

In another aspect the invention provides a method of producing a suspension of liposomes comprising extruding an aqueous suspension of lipids through a hydrophilic, angled pore screen membrane at high pressure.

In another aspect the invention provides a device for extruding an aqueous suspension of lipids at high pressure comprising a hydrophilic screen membrane(s) in a support cassette holder.

In practicing the methods of the invention, a suspension of lipids is extruded through a hydrophilic screen membrane at high pressure. The resulting liposomes have an average diameter of between about 50 and 400 nm and a standard size distribution of about 50 nm depending on the number of membranes used, the number of times the liposomes are cycled through the membranes, the thickness of the membranes, the pressure of the extrusion, the diameter and density of pores in the membranes, the chemical composition of the membranes, use of a step-down method, the type of lipid used, the use of a wetting agent, the presence of a liposome-encapsulated agent or a liposome-associated agent, etc., as will be described in detail below. The methods of the invention allow one to control the size and size distribution of the liposomes produced.

The suspension may be passed through the membrane multiple times, each time in the same direction through the membrane. Alternatively, the direction of flow through the membrane can be reversed for one or more of the passes. That is, the suspension may be passed through the membrane in an outside-to-inside direction to maintain the membrane in an unclogged condition, allowing high throughput processing, even for a concentrated suspension of liposomes.

The desired liposome size may be achieved using a “step-down” method. That is, a suspension of lipids is passed through membranes of decreasing pore diameter size in a series of passes in order to produce liposomes of increasingly reduced average diameter.

These and other aspects and features of the present invention will become more fully apparent when the following detailed description of the invention is read in conjunction with the accompanying drawings.

### BRIEF DESCRIPTION OF THE DRAWINGS

FIG. 1 presents extrusion volume as a function of time for two different commercially available membranes (0.1 μm pore diameter polycarbonate track-etched (PCTE) membranes from Osmonics (“O”) and Whatman (“W”)) at 400 and 800 psi.

FIG. 2 is a set of graphs that compare extrusion through a single PCTE membrane to a single polyester track-etched (PETE) membrane, both membranes having 0.1 μm pore diameters. FIG. 2A is a graph presenting particle size as a function of pass number. FIG. 2B is a graph presenting flow rate as a function of pass number.

FIG. 3A is a graph comparing the extruded volume versus time for extrusion of 20% 1-palmitoyl-2-oleoyl-sn-glycero-3-phosphocholine (POPC) through 0.1 μm avg. pore diameter PCTE and PETE membranes at 400 psi. FIG. 3B is a graph comparing the extruded volume versus time for extrusion of 20% POPC through 0.1 itm avg. pore diameter PCTE and PETE membranes at 800 psi.

FIG. 4 is a series of graphs describing the effects of membrane stack size on the extrusion of POPC using 1-, 2-, 5-, and 10-stacked 0.1 μm average pore diameter PETE membranes at 800 psi. FIG. 4A is a graph presenting average large unilamellar vesicle (LUV) particle size as a function of pass number. FIG. 4B is a graph presenting flow rate as a function of pass number. FIG. 4C is a graph relating membrane stack size to the number of passes needed to achieve an average particle size of about 120 nm.

FIG. 5 is a series of graphs describing the effects of pressure on the extrusion of POPC using a 5-stack of 0.1 μm pore diameter PETE membranes at extrusion pressures of 400, 600 and 800 psi. FIG. 5A is a graph presenting particle size as a function of pass number. FIG. 5B is a graph presenting flow rate as a function of pass number. FIG. 5C is a graph relating extrusion pressure to the number of passes needed to produce particles with an average size of about 120 nm.

FIG. 6 presents maximum extrusion volume as a function of extrusion pressure for 0.1 μm Osmonics PORETICS™ PCTE and PETE and Whatman NUCLEPORE™ PCTE membranes before membrane fouling or clogging.

### DETAILED DESCRIPTION OF THE INVENTION

In one aspect, the present invention provides a simple and fast method for producing vesicles of a desired size by extruding a preparation of lipids through a hydrophilic screen membrane at high pressure. The preparation of lipids can be extruded through the membrane once or more than once, in multiple “passes,” to produce vesicles of the desired size. When multiple passes are used, the direction of flow of suspension through the membrane can be reversed for one or more of the passes. The preparation of lipids also can be extruded through a plurality of “stacked” membranes to decrease the number of passes required to achieve particles of the desired size. Alternatively, the preparation of lipids can be passed within the same pass through consecutively arranged membranes that are stacked, un-stacked, or a combination of stacked and un-stacked in their consecutive arrangement.

In another aspect, the present invention provides a device for producing liposomes of between about 50 and about 400 nm in diameter by extruding an aqueous suspension of lipids through a hydrophilic screen membrane at high pressure.

An important feature of the methods and devices of the present invention is the use of high pressure to drive the aqueous suspension of lipids through the membrane. It has been found that the use of pressures greatly in excess of the minimum pressure required for extrusion provide unexpectedly good results in achieving liposomes of the desired average diameter and avoiding problems associated with membrane clogging or fouling. The use of high pressure is especially beneficial for the extrusion of lipids that are difficult to extrude using conventional methods. As shown in the examples, the greater the pressure used, the less clogging and fouling occurs. There is no apparent upper limit to this relationship. The pressure used is limited only by the tolerance of the extrusion device, the membrane supports and the membranes used. At a minimum, the pressure used should be greater than about 400 psi. Preferably, a pressure of greater than about 800 psi is used. More preferably, a pressure of greater than about 1,000 psi is used. More preferably still, a pressure in excess of about 1,500 psi is used. More preferably still, a pressure in excess of about 5,000 psi is used. Most preferably, a pressure in excess of about 8,000 psi is used.

As used herein, “liposome”, “vesicle” and “liposome vesicle” will be understood to indicate structures having lipid-containing membranes enclosing an aqueous interior. The structures may have one or more lipid membranes unless otherwise indicated, although generally the liposomes will have only one membrane. Such single layered liposomes are referred to herein as “unilamellar”. Unilamellar liposomes can be classified as being small unilamellar vesicles (SUVs), large unilamellar vesicles (LUVs) or giant unilamellar vesicles (GUVs). See, Lasic, 1997, Liposomes in Gene Delivery, CRC Press LLC, Boca Raton 67-71 at page 70. See, e.g., Frisken, 2000, Langmuir 16, 928-33; Hunter, et al., 1998, Biophys. J. 74: 2996-3002. SUVs are typically defined as liposomes wherein curvature effects are important for their properties. Using this definition, the size of a liposome that can be characterized as an SUV will depend on the lipid or lipids it comprises. Generally, for a soft bilayer, the upper limit for an SUV is about 50 nm, whereas for mechanically very cohesive bilayers, the upper limit can range from about 80 to about 100 nm. GUVs typically are defined as liposomes with diameters greater than about 1 μm. One of skill in the art will understand that the boundaries between these classes of vesicles are not sharply defined and that there is considerable overlap between them at their margins.

In one embodiment, the liposomes of the invention can be lipid-containing membranes enclosing an aqueous interior, the aqueous interior containing a drug compound. In another embodiment, the liposome can contain no drug in the aqueous interior, but are the lipid-containing membrane enclosing an interior medium. Such non-drug containing liposomes are useful for removing cholesterol from the blood stream and treating or preventing atherosclerosis.

As used herein, “bound to the liposome” or “binding to the liposome” indicates that the subject compound is covalently or non-covalently bound to the surface of the liposome or contained, wholly or partially, in the interior of the liposome.

The terms “pharmaceutically active compound” and “drug” are meant to indicate a synthetic compound suitable for therapeutic use without associated bound carriers, adjuvants, activators, or co-factors. The liposomes of the invention can contain a drug in the aqueous interior. In certain embodiments, the liposomes can contain no drug in the interior and in such embodiments, the liposomes themselves can be drugs or pharmaceutically active compounds. These ‘empty’ liposomes can be useful for the removal of cholesterol from the body and treating or preventing atherosclerosis.

“Screen membranes” are membranes that have pores whose channels through the membrane on average exhibit essentially straight lines. A screen membrane can have pores that are normal to the plane of the membrane and/or angled pores. “Angled pore membranes” are screen membranes wherein the angles formed by the pores relative to the plane of the face of the membrane is less than about 90°. The “reef length” of a pore is the length of the pore measured from one membrane face to the other. Thus, a pore that is normal to the plane of the membrane has a reef length that is equal to the thickness of the membrane. Pores with smaller pore angles have greater reef lengths.

For a given droplet on a solid surface, the “contact angle” is a measurement of the angle formed between the surface of a solid and the line tangent to the droplet radius from the point of contact with the solid. The contact angle is related to the surface tension by Young's equation through which the behavior of specific liquid-solid interactions can be calculated. A contact angle of zero results in wetting, while an angle between 0° and 90° results in spreading of the drop (due to molecular attraction). Angles greater than 90° indicate the liquid tends to bead or shrink away from the solid surface. Thus, the smaller the contact angle between a surface and a water droplet, the more hydrophilic the surface. See, e.g. Martin, et al., 1983, Physical Pharmacy: Physical Chemical Principles in the Pharmaceutical Sciences, Lea & Febiger Publishers, Philadelphia; Gennaro, et al., 1990, Remington's Pharmaceutical Sciences, 18^{th} edition, Mack Publishing Company, Easton, Pa.

“Difficult lipids” are lipids that are relatively difficult to extrude using conventional techniques because they tend to clog or foul the extrusion membrane. Conversely, “easy lipids” are lipids that are relatively easy to extrude using conventional techniques. Typically, lipids having a transition temperature (T_{c}) greater than about room temperature are difficult to extrude, while lipids having a T_{c} of about room temperature or lower are easy to extrude. A few difficult lipids have transition temperatures below room temperature, e.g., 1-palmitoyl-2-oleoyl-sn-glycero-3-phosphocholine (POPC). A number of different factors can influence whether a particular lipid is difficult to extrude. The most important factor is the rigidity of the lipid's acyl chain. Lipids having more rigid acyl chains, for example, lipids comprising a mono-unsaturated acyl chain, tend to be more difficult to extrude using conventional methods and devices. Impurities in the lipid preparation, e.g., resins introduced during the lipid manufacturing process, also can make a lipid more difficult to extrude. In addition, impurities can affect the conformation of lipids in solution or the lipid's ability to deform through membrane pores. These problems can be overcome if membranes with larger surface area or greater porosity are used, e.g. Whatman ANOPORE™ membranes. Drug-associated lipids, charged lipids and lipids with proteins also can be difficult lipids. Some lipids that are easy to extrude on a laboratory scale can be difficult to extrude on a larger manufacturing scale. Examples of difficult to extrude lipids include, but are not limited to, POPC, dipalmitoylphosphatidylcholine, dipalmitoylphosphatidylglycerol and di-stearoyl-phosphatidylethanolamine. Examples of easy lipids include, but are not limited to, egg yolk phosphatidylcholine (EPC), egg phosphatidylglycerol and di-oleyl-phosphatidylcholine.

Membranes

A membrane useful for practicing the present invention is a hydrophilic screen membrane. A hydrophilic screen membrane useful for practicing the claimed invention can be made from any hydrophilic material. The membrane can be made out of a single hydrophilic material, or more than one hydrophilic material, or a mixture of hydrophilic and non-hydrophilic materials. In a preferred embodiment, the membrane is made of a naturally hydrophilic material. In another preferred embodiment the membrane is made of material that is made hydrophilic during membrane production, e.g., polyester. In a preferred embodiment, hydrophilic screen membranes useful for practicing the present invention have a surface water contact angle of less than 120 degrees, preferably less than 70 degrees, more preferably less than 50 degrees, most preferably 40 degrees or less. In another preferred embodiment, hydrophilic screen membranes useful for practicing the claimed invention have a surface tension prior to etching of about 41 dynes/cm or greater, preferably 42 dynes/cm or greater, and most preferably 43 dynes/cm or greater. Specific hydrophilic membranes useful for practicing the present invention include, but are not limited to, polyethylene terephthalate (polyester), aluminum oxide, polyacrylonitrile, cellulose acetate, cellulose mixed ester, glass, polyethersulfone, polysulfone and poly hexamethylene adipamide (nylon). It should be noted that we have determined that polyester is currently the most suitable commercially available membrane for use within the methods and devices of the invention.

Membranes made from more hydrophobic materials also can be used, provided that they are modified to exhibit more hydrophilic properties. Methods of increasing the hydrophilicity of membranes are well known in the art, and include, but are not limited to, treating the membrane with a surfactant, coating the membrane with a wetting agent or thin film application of a different polymer or monomer-system to form a new surface via composite formation, e.g., polyvinyl pyrolidine (PVP), chemical grafting of a low molecular weight active group (monomer) to the membrane, forming the membrane from a combination of two or more polymers and plasma modification. See, e.g. Martin, et al., 1983, Physical Pharmacy: Physical Chemical Principles in the Pharmaceutical Sciences, Lea & Febiger Publishers, Philadelphia; Gennaro, et al., 1990, Remington's Pharmaceutical Sciences, 18^{th} edition, Mack Publishing Company, Easton, Pa.

In another preferred embodiment, a screen membrane useful for practicing the present invention is made from TEFLON® (Du Pont, Wilmington, Dela.). In a more preferred embodiment, the membrane consists of or consists essentially of TEFLON®. In another more preferred embodiment, the membrane comprises TEFLON® and one or more other substances.

Screen membranes have “straight through” channels (also called capillary-type pores). That is, the pore channel exhibits or describes a substantially straight line through the membrane. If this line is perpendicular, or normal, to the plane of the face of the membrane, then it has a pore angle of 90°. Angled pore membranes have a pore angle of less than about 90°.

Screen membranes manufactured using any method can be used in the methods and devices of the present invention. Screen membranes typically are manufactured in a two step track etching procedure, see, e.g.,Wagner, 2001, Membrane Filtration Handbook: Practical Tips and Hints, 2^{nd} Edition, Printed by Osmonics, Inc., Minnetonka, Minn. In the first step, the membrane is exposed to ionizing radiation. This radiation forms damage tracks that are randomly distributed across the face of the membrane. The damage tracks are etched into pores through the membrane by immersing the membrane into an etching solution, for example, a strong alkaline solution such as sodium hydroxide. The angle at which the charged particles strike and pass through the membrane in the first step determines the pore angle of the resulting channel. Thus, one can produce a filter with a desired average pore angle by controlling the angle at which the charged particles strike the membrane.

The etching process can affect the hydrophilicity of the membrane. For many types of membranes, including polycarbonate and polyester membranes, immersion in the etching bath increases the hydrophilicity of the membranes. Some membranes, such as polyester membranes, become more hydrophilic more quickly than other membranes, such as polycarbonate membranes, treated under similar etching conditions. See, e.g., Kroschwitz, 1990, Concise Encyclopedia of Polymer Science and Engineering, Wiley, N.Y., 363-67, 558-60; Domininghaus, 1993, Plastics for Engineers: Material, Properties, Applications, Hanser Publishers, New York, Chapter 14; Zeronian, et al., 1990, J. Appl. Polym. Sci. 41:527-34; Gillberg, et al., 1981, J. Appl. Polym. Sci. 26:2023-51.

We have found that extrusion through Osmonics PORETICS™ membranes results in greatly increased flow rates compared to extrusion through other commercially available membranes, and that this difference is particularly great when the extruded lipid is a difficult lipid. Without being bound to a particular theory, we note that flow rate and liposome particle size are influenced by a number of factors, including, but not limited to, pore diameter, pore density, average pore angle, range of pore angles, membrane thickness and the material used to make or coat the membrane, as described herein. The properties of polyester, polycarbonate and other membranes are discussed at, for example, Kroschwitz, 1990, Concise Encyclopedia of Polymer Science and Engineering, Wiley, N.Y.; Domininghaus, 1993, Plastics for Engineers: Material, Properties, Applications, Hanser Publishers, New York; Zeronian, et al., 1990, J. Appl. Polym. Sci. 41:527-34 and Gillberg, et al., 1981, J. Appl. Polym. Sci. 26:2023-51.

In a preferred embodiment, the membranes are angled pore membranes. In a more preferred embodiment, the pores of the membrane have an average angle relative to the plane of the face of the membrane (i.e., an average pore angle) of less than about 56°. In a most preferred embodiment, the average pore angle is about 45°. In another preferred embodiment the average pore angle is about 90° in order to minimize the reef length of the pore to the same distance as the membrane's thickness.

Commercially available polyester membranes suitable for use in the present invention include, but are not limited to, NUCLEPORE™ PETE membranes, Cat. No.s 188607, 188107, 188606, 188106, 188605, 188105 and 188604 (Whatman), CYCLOPORE™ PETE membranes, Cat. No.s 7061-2504, 7061-4704, 7061-2502, 7061-4702, 7061-2501 and 7061-4701 (Whatman) and PORETICS™ PETE membranes, Cat. No.s T01CP02500, T04CP04700, T02CP02500, T02CP04700, T01CP02500 and T01CP04700 (Osmonics).

A membrane of any thickness can be used in the methods and devices of the present invention. One of skill in the art will appreciate that a thicker membrane produces smaller vesicles and has a slower flow rate compared to a thinner membrane under otherwise similar conditions. The upper size limit of the thickness of the membrane useful in the methods and devices of the present invention is determined by the tolerance of the extrusion device used. The lower limit of the thickness of the membrane useful in the methods and devices of the present invention is determined by the fragility of the membrane and its ability to withstand the pressure of extrusion. In a preferred embodiment, the membrane is between about 3 and about 50 μm in thickness. In a more preferred embodiment, the membrane is between about 3 and about 20 μm in thickness. In a most preferred embodiment, the membrane is between about 3 and about 12 μm in thickness.

A membrane of any size and shape can be used in the methods and devices of the present invention. The size and shape of the membrane is limited only by the tolerance of the extrusion device. In general, the larger the surface area of the membrane, the greater the flow rate through the membrane. A membrane of any desired size or shape can be cut from a larger sheet of membrane. The membrane can be, for example, circular, square or rectangular with a surface area of about 1 cm² to about 3 m². In a preferred embodiment, the membrane is circular and has a diameter of about 25 mm. In another preferred embodiment, the membrane is circular and has a diameter of about 47 mm. In another preferred embodiment, the membrane is circular and has a diameter of about 90 mm. In another preferred embodiment, the membrane is circular and has a diameter of about 142 mm. In yet another preferred embodiment, the membrane is circular and has a diameter of about 293 mm.

A membrane of any topology can be used in the methods and devices of the present invention, limited only by the tolerance of the extrusion device employed. One of skill in the art knows how to manipulate the topology of the membrane to increase the surface area of the membrane that is in contact with the preparation to be extruded, and that this reduces clogging or fouling of the membrane. In a preferred embodiment, the membrane is flat. In another preferred embodiment, the membrane is pleated.

A membrane with any average pore diameter can be used in the methods and devices of the present invention. A membrane with a larger average pore diameter will produce larger vesicles, and have a greater flow rate, than a membrane with a smaller average pore diameter under otherwise similar conditions. In a preferred embodiment, the membrane has an average pore diameter that is approximately equal to the diameter of the vesicles to be produced. In another preferred embodiment, the average pore diameter is between about 50 and about 400 nm. In a more preferred embodiment, the average pore diameter is between about 75 and about 200 nm. In a still more preferred embodiment, the average pore diameter is between about 100 and about 125 nm. In a most preferred embodiment, the average pore diameter is about 100 nm.

A membrane with any pore density can be used in the methods and devices of the present invention. A membrane with a greater pore density will have less fouling or clogging and a greater flow rate than a membrane with a lesser pore density under otherwise similar conditions. Thus, in general, a greater pore density is preferred. However, large pore densities are associated with several drawbacks. First, a high pore density can compromise the tensile strength of the membrane, thus compromising its ability to withstand the extrusion pressure it is subjected to. Second, in a membrane with randomly-distributed pores, the number of overlapping pores increases with increasing pore density. Overlapping pores have pore diameters greater than the rated size of the membrane, thus they can cause an increase in the average diameter of the vesicles extruded by the membrane. Consequently, a pore density is preferred that is as high as can be achieved without compromising the tensile strength of the membrane or significantly increasing the average particle diameter of the extruded vesicles. The maximum achievable or desirable pore density also is limited by the average pore diameter of the membrane. A membrane with a larger average pore diameter has a lower maximum pore density than an otherwise similar membrane with a smaller average pore diameter. In a preferred embodiment, the pore density is between about 8×10⁵ and 9×10⁹ randomly distributed pores/cm². In a more preferred embodiment, the pore density is between about 8×10⁶ and 5×10⁹ randomly distributed pores/cm². In a most preferred embodiment, the pore density is between about 1.5x10⁷ and 2.6x10⁹ randomly distributed pores/cm².

Greater pore densities can be achieved in a membrane without significantly increasing its average pore diameter by using a non-random distribution of pores. A screen membrane with a patterned array of pores can be created wherein the incidence of overlapping pores is virtually eliminated, even at very high pore densities. Thus, in a preferred embodiment, the pore density is between about 8×10⁵ and 9×10⁹ non-randomly distributed pores/cm². In a more preferred embodiment, the pore density is between about 8×10⁶ and 5×10⁹ non-randomly distributed pores/cm². In a most preferred embodiment, the pore density is between about 1.5×10⁷ and 2.6×10⁹ non-randomly distributed pores/cm².

An apparatus for efficiently handling and/or replacing membranes during the extrusion process can be used with the methods of the invention. Such an apparatus can be a support holder comprising, for example, a membrane(s) and a support ring(s) for holding the membrane(s) in a single plane to prevent folding or sticking. The support holder apparatus can be comprised of membranes of different or similar pore size diameter, arranged in a sandwich or stacked configuration. The apparatus of the invention can provide ease and convenience when working with membranes, for example, the apparatus can be easily removed or replaced during the extrusion process and can be sterilized.

Methods of the Invention

According to the methods of the invention, a material capable of forming a vesicle, micelle or liposome is extruded through a screen membrane at high pressure to produce a suspension of vesicles, micelles or liposomes. Exemplary materials suitable for extrusion using the methods and devices of the present invention are discussed below.

The methods and devices of the present invention are practiced using a high extrusion pressure. An extrusion conducted at a higher pressure will have a higher flow rate, clog or foul less readily, allow the membrane to tolerate a greater degree of fouling or clogging during production, and produce vesicles of a smaller size than an otherwise identical extrusion conducted at a lower pressure. The pressure that can be used is limited only by the tolerance of the extrusion device and the membrane used. In a preferred embodiment, a pressure of greater than about 400 psi is used. In another preferred embodiment, a pressure of greater than about 800 psi is used. In a more preferred embodiment, a pressure of greater than about 1,500 psi is used. In a still more preferred embodiment, a pressure of greater than about 5,000 psi is used. In a most preferred embodiment, a pressure of greater than about 8,000 psi is used in the invention.

The present invention can be practiced at any temperature. In a preferred embodiment, the extrusion is conducted at a controlled temperature. In a more preferred embodiment, the controlled temperature is a constant temperature. In another embodiment, the constant temperature is about room temperature. In another embodiment, the constant temperature is equal to or greater than the T_{c} of the lipid being extruded. In another embodiment, the mixture being extruded comprises a plurality of lipids, and the constant temperature is equal to or greater than the highest T_{c} of the lipids being extruded. In another embodiment, the constant temperature is between about 15° C. and about 35° C. In a more preferred embodiment, the constant temperature is between about 20° C. and about 30° C. In a more preferred embodiment, the constant temperature is between about 23° C. and about 27° C. In a most preferred embodiment, the constant temperature is about 25° C.

The methods and devices of the present invention can be used to make vesicles of any desired average diameter. Generally, a membrane is selected that has an average pore diameter similar to the desired average vesicle diameter, as explained above. The average vesicle size can be reduced by, for example, extruding the extruded vesicles one or more additional times, using a stack of membranes, using a thicker membrane, increasing the pressure of extrusion or processing the vesicles, as described herein. The size of the vesicles may be determined using any technique known in the art. For example, quasi-electric light scattering (QELS), also known as Dynamic Light Scattering (DLS), can be used as described in Bloomfield, 1981, Ann. Rev. Biophys. Bioeng. 10:421-50. In a preferred embodiment, the vesicles have an average diameter of between about 50 and 400 nm. In a more preferred embodiment, the average diameter is between about 50 and 150 nm. In a still more preferred embodiment, the average diameter is between about 100 and 150 nm. In a-most preferred embodiment, the average diameter is about 169±37 nm, 158±39.5 nm, 136±42 nm, 153.6±45.2 nm, 138.6±35.6 nm, 114.4±35.8 nm or 118.1±36.2 nm.

The methods and devices of the present invention can be used to make vesicles of a desired lamellarity. A unilamellar vesicle has a single layer of membrane. A multilamellar vesicle (MLV) comprises a plurality of membrane layers. See, Lasic, 1997, Liposomes in Gene Delivery, CRC Press LLC, Boca Raton 67-71. In a preferred embodiment of the invention, a suspension of MLVs is extruded using the methods or devices of the present invention to produce a suspension of unilammelar vesicles of a desired average diameter. In another preferred embodiment, an emulsion is extruded using the methods or devices of the present invention.

Vesicles produced using the methods or devices of the present invention can be further processed using any processing technique. In a preferred embodiment, the average vesicle diameter of a suspension of vesicles produced using the methods or devices of the present invention is altered after they are extruded. In a more preferred embodiment, the extruded vesicles are extruded one or more additional times. In a still more preferred embodiment, the additional extrusion is done using the methods or devices of the present invention. In a most preferred embodiment, the vesicles are extruded in multiple passes using a “step-down” procedure, i.e., wherein each successive extrusion is through a membrane of smaller average pore diameter. In another more preferred embodiment, the suspension is passed through the membrane alternately in the forward and reverse directions to reduce the amount of clogging or fouling of the membrane.

In another preferred embodiment, the average diameter of the extruded vesicles is reduced further by sonication. In another preferred embodiment, intermittent sonication cycles are alternated with QELS assessment to guide efficient vesicle synthesis.

In another preferred embodiment, the extruded vesicles are processed in order to remove contaminants or impurities. In another preferred embodiment, the suspension to be extruded contains a substance to be incorporated into the vesicles, and the processing step removes that portion of the substance that was not incorporated into the vesicles. In a more preferred embodiment, the substance to be incorporated into the vesicles is a pharmaceutically active substance, such as a small molecule drug, protein, peptide, nucleic acid or oligonucleotide.

The methods and devices of the present invention can be practiced using any number of stacked membranes. One of skill in the art appreciates that an extrusion through a greater number of stacked membranes will have a lower flow rate and produce vesicles having a smaller average diameter than an otherwise similar extrusion through a smaller number of stacked membranes. The number of membranes that can be used in a stack is limited only by the tolerance of the extrusion device. In a preferred embodiment, the stack comprises between 2 and 10 membranes. In a most preferred embodiment, the stack comprises between 2 and 5 membranes. In another preferred embodiment, the stacked membranes are essentially identical. In another preferred embodiment, at least one of the membranes in the stack is different from at least one of the other membranes in the stack. The difference can be in any property that affects the extrusion. The difference can be, for example, in the composition of the membrane, coating, pore size, pore density, pore angle, pore shape or membrane size, as described herein.

In another preferred embodiment, the extrusion is performed using multiple passes through a membrane or stack of membranes. If a stacked membrane embodiment is used in the extrusion, multiple passes may not be necessary in order to achieve liposomes of a desired diameter. In a particularly preferred embodiment, a step-down method is employed. In a step down method, multiple passes of the suspension are done through membranes of decreasing pore diameter. In a particularly preferred embodiment of the step-down method, a first pass is done through a membrane with a pore diameter of about 0.4 μm, a second pass is done through a membrane with a pore diameter of about 0.2 μm, and if necessary, a third, a fourth, a fifth and a sixth pass are done through a membrane with a pore diameter of about 0.1 μm.

In another preferred embodiment, the membrane is treated with a flushing agent. In a more preferred embodiment, the membrane is treated with the flushing agent prior to the extrusion. In another more preferred embodiment, the membrane is treated with the flushing agent after at least one pass through the membrane has been completed and prior to at least one more pass being performed through the membrane. The flushing agent can be any substance or composition that removes material from a clogged or fouled membrane pore or prevents membranes from clogging or fouling or creating a “sieving effect”. In a preferred embodiment, the flushing agent comprises an organic alcohol. In a more preferred embodiment, the flushing agent comprises ethanol.

Extrusion Devices

Any extrusion device capable of housing an appropriate membrane and withstanding a high extrusion pressure can be used to practice the methods and devices of the claimed invention. In a preferred embodiment, the extrusion device of the present invention and devices useful for practicing the methods of the present invention comprise a hydrophilic, angled pore or hydrophilic angled pore screen membrane. In a more preferred embodiment, the membrane is a polyester track-etched (PETE) membrane. In another more preferred embodiment, the extrusion device additionally comprises a housing and a collection vessel, wherein the housing is operably attached to a first side of the membrane by a pressure- and liquid-resistant seal and the collection vessel is positioned to receive the extruded suspension after it exits a second side of the membrane. In a still more preferred embodiment, the device additionally comprises a membrane support or apparatus. In another preferred embodiment, the extrusion device is configured such that the aqueous suspension can alternately be extruded through the membrane in the forward and reverse directions. In another preferred embodiment, the extrusion device uses a tangential flow. Commercially available devices that can be fitted with the appropriate membranes and used in the present invention include, but are not limited to, THE MINI-EXTRUDER™, Cat. No. 610000 (AVANTI® Polar Lipids, Inc., Alabaster Ala.), see, Subbarao, et al., 1991, Biochim. Biophys. Acta, 1063:147-54, Liposome Extruder, Part No. ER-1 (Eastern Scientific, Rockville Md.), see, EMULSIFLEX®-C50 Extruder, Cat. No. EFC50EX (Avestin, Inc., Ottowa, Ontario, Canada), see, LIPOSOFAST™, (Avestin, Inc.), LIPEX™ Extruders (Northern Lipids Inc., Vancouver, British Columbia, Canada). Other extrusion devices useful for practicing the present invention include those described in U.S. Pat. Nos. 5,948,441; 5,556,580 and 6,217,899 B1.

The extrusion device must be capable of withstanding high extrusion pressures. As a general rule, greater pressures result in improved performance, for example, increased flow rates, less membrane fouling and clogging and a more rapid reduction in size of the extruded vesicles. At a minimum, the extrusion device should be capable of withstanding extrusion pressures greater than about 400 psi. In a preferred embodiment, the extrusion device can withstand an extrusion pressure greater than about 800 psi. In a more preferred embodiment, the extrusion device can withstand an extrusion pressure greater than about 1,000 psi. In another more preferred embodiment, the extrusion device can withstand an extrusion pressure greater than about 1,500 psi. In a more preferred embodiment, the extrusion device can withstand an extrusion pressure of greater than about 5,000 psi. In a most preferred embodiment, the extrusion device can withstand an extrusion pressure of greater than about 8,000 psi.

A membrane support holder or housing also can be used that optimize the available surface area, provided that it can withstand the extrusion pressure that it is subjected to. In a preferred embodiment, the membrane is pleated. In another preferred embodiment, the support holder or housing utilizes three-dimensional membrane positioning. The invention further provides methods and an apparatus for efficiently handling membranes and/or replacing membranes during an extrusion process. Commercially available polycarbonate track-etched (PCTE) and polyester track-etched (PETE) membranes are flimsy in nature and statically charged. These membranes bend easily and stick to themselves making them difficult to handle and position into membrane holders used for extrusion. This is especially apparent when the membranes or membrane holders are wet. In addition, PCTE, PETE and other types of membranes are very delicate and require careful handling. To ease this problem, the invention provides methods and an apparatus to efficiently enable handling and loading of the membranes into holders by use of a membrane support holder or housing. A membrane support holder can be, for example, a cartridge support holder.

In one embodiment, the cartridge support or holder can be comprised of a support ring(s) capable of fixing the membrane or membranes to the perimeter of the support ring(s) in a single plane to prevent folding and sticking at high pressures. The apparatus is capable of holding the membrane or membranes in various configurations, for example, the membranes can be housed in a ‘sandwich’ or in a stacked configuration. If multiple membranes are housed in the cartridge, the membranes can be of the same or different pore size diameter. The apparatus or cartridge can have a variety of other features, for example, the cartridge support holder can be pre-loaded with membranes, the apparatus can be sterilized and easily incorporated into an automated membrane change-out system.

The cartridge holder apparatus provides the advantages of minimizing membrane handling and greater efficiencies in loading the membranes. In addition, the cartridge holder apparatus provides overall improved efficiency in production because, for example, a clogged or fouled membrane can be changed while product flow is diverted to a new cartridge holder apparatus while the clogged or fouled cartridge holder apparatus is changed out.

Lipids

The methods and devices of the present invention can be used to extrude vesicles, micelles or liposomes from any suitable substance. In a preferred embodiment, the methods and devices of the present invention are used to produce liposomes from a lipid or combination of lipids. Any lipid or combination of lipids can be used. In a preferred embodiment, the extruded lipid is difficult to extrude using conventional methods and devices. In a more preferred embodiment, the difficult lipid has a T_{c} greater than about room temperature. In another preferred embodiment, the difficult lipid comprises a rigid acyl chain. In a more preferred embodiment, the rigid acyl chain is a mono-unsaturated acyl chain. In another preferred embodiment, the difficult lipid comprises an impurity or contaminant. In a more preferred embodiment, the impurity or contaminant is a resin or impurity which makes the lipid difficult to extrude. In a more preferred embodiment, the impurity or contaminant is a resin introduced during the manufacturing process. In another preferred embodiment, the difficult lipid is associated with another molecule. In a more preferred embodiment, the molecule is a drug. In another more preferred embodiment, the molecule is a protein. In another preferred embodiment, the difficult lipid is a charged lipid. In another preferred embodiment, the difficult lipid is difficult to extrude on a manufacturing scale. In a particularly preferred embodiment, the difficult lipid is selected from the group consisting of 1-palmitoyl-2-oleoyl-sn-glycero-3-phosphocholine (POPC), dipalmitoylphosphatidylcholine, dipalmitoylphosphatidylglycerol and di-stearoyl-phosphatidylethanolamine.

In another preferred embodiment, the lipid is an easy lipid. In a more preferred embodiment, the easy lipid is selected from the group consisting of egg yolk phosphatidylcholine (EPC), egg phosphatidylglycerol and di-oleoyl-phosphatidylcholine.

Other phospholipids suitable for use with the present invention include, but are not limited to, di-lauroylphosphatidylcholine, di-lauroylphosphatidylglycerol, oleoyl-palmitoylphosphatidylcholine, glycolipid-linked phospholipdis, phosphatidylcholine, phosphatidylglycerol, lecithin, β, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, and the like.

In a most preferred embodiment, the lipid is phosphatidylcholine or sphingomyelin.

Non-phosphorus containing lipids also may be used in the liposomes of the compositions of the present invention. These include, but are not limited to, cholesterol, other sterols, stearylamine, docecylamine, acetyl palmitate and fatty acid amides.

Additional lipids suitable for use in the liposomes of the present invention are well known to persons of skill in the art and are cited in a variety of well known sources, e.g., McCutcheon's Detergents and Emulsifiers and McCutcheon's Functional Materials, Allured Publishing Co., Ridgewood, N.J.

Lipids used in the methods and devices of the present invention include chemically modified lipids. In a preferred embodiment, the lipid is covalently attached to a modifying group. The modifying group can affect any property or properties of the lipid. For example, the modifying group can alter the lipid's transition temperature, assembly properties, extrusion properties, encapsulation properties, in vivo targeting properties, in vivo processing properties, physiological effects, stability or half life. In a preferred embodiment, the modified lipid is polyethylene glycol-linked (PEG-linked). In another preferred embodiment, the modified lipid is a PEGylated phospholipid.

In another preferred embodiment, combinations of lipids can be used in the methods and devices of the present invention. For example, a phospholipid and PEG-linked lipid can used in the methods and devices of the invention.

The preparation to be extruded also can contain other types of molecules. Examples of other molecules or ions that can be associated with the lipids include, but are not limited to, cholesterol or other steroids or steroid derivatives, solvents, buffers, acids, bases, salts, metals, chelators, sugars, proteins, nucleic acids and drugs, as described below. See, e.g., Lasic, 1997, Liposomes in Gene Delivery, CRC Press LLC, Boca Raton 67-71.

For the preparation of liposomes to be administered to a subject, it is generally desirable that the liposomes be composed of lipids that are liquid-crystalline at 37° C., often at 35° C., and even 32° C. As subjects typically have a core temperature of about 37° C., liposomes composed of lipids that are liquid-crystalline at 37° C. are generally in a liquid-crystalline state during treatment.

The highest quality of raw materials is used in the methods, in part because of the high pressures that are employed. The raw material lipids should meet certain standards of quality control before being used in the extrusion process. For example, pH, powder size, form of dried powder, wetted particle size, osmolality, calcium level, particulate level, drying conditions and levels of additives, residuals or impurities from the production process should be controlled. These parameters may affect the physical characteristics of lipids in solution or suspension making the lipids difficult to extrude. In particular, the pH should be controlled and consistent, the calcium levels should be low and the raw material should dry well and have good visual characteristics.

Preparation of Lipids

Any preparation comprising one or more substances that can be extruded to form vesicles, micelles or liposomes can be used in the methods and devices of the present invention. In a preferred embodiment, a preparation comprising one or more lipids is used. In a particularly preferred embodiment, the preparation is an aqueous suspension comprising one or more lipids. Any method of making such a preparation can be used. See, e.g., Lasic, 1997, Liposomes in Gene Delivery, CRC Press LLC, Boca Raton 67-71 at pages 88-91; Szoka, et al., 1980, Biochim. Biophys. Acta, 601:559-71. These methods generally involve making an aqueous suspension of lipids. In a preferred embodiment, the lipids form multilamellar vesicles (MLVs) in the suspension. Suspensions of MLVs can be extruded to produce vesicles of the desired size and lammellarity, for example, SUVs or LUVs. Typically, a lipid concentration of between about 5 and 50 mM is used, although lipid concentrations of up to about 400 mg/ml or greater are feasible. Where a plurality of lipids is used, the lipids generally are first mixed in an organic solvent, such as chloroform, a 3:1 (v:v) chloroform:methanol mixture or tertiary butanol. The lipids are dissolved in the solvent, typically at a temperature of between about 30° C. to about 50° C., then rapidly frozen, for example, by incubating in a dry ice-ethanol or dry ice-acetone bath. The organic solvent is then evaporated, and the dry lipid film, cake or powder is rehydrated in an appropriate aqueous solution. Rehydration is typically conducted at a temperature greater than the T_{c} of the lipid with the highest T_{c} (if more than one lipid is used) in an aqueous solution, for example, distilled water, buffered distilled water, saline solution, or a sugar solution or other solution of dissolved nonelectrolytes. The hydration step preferably lasts longer than about 1 hour and is accompanied by agitation, although it can be accomplished in as little as a few minutes, depending on the lipid. The size range of MLVs formed during the hydration process generally range from about 500 nm to about 10,000 nm (10 microns) or greater. Typically, more vigorous agitation during hydration favors the formation of smaller MLVs. Hydration is optionally followed by allowing the mixture to rest undisturbed overnight, which can facilitate the subsequent formation of unilammelar vesicles. In a preferred method of producing an aqueous suspension of lipids, a chloroform solution of lipid is vortexed and the solvent removed under a steady stream of N₂. The sample is dried under a high vacuum. The resulting dry lipid film is rehydrated in 150 mM NaCl and 20 mM [4-(2-hydroxyethyl)]-piperazine-ethanesulfonic acid (Hepes, pH 7.4).

In another preferred embodiment, the preparation to be extruded comprises an emulsion of one or more lipids. The emulsion can be formed using any known technique and mechanical device such as an homogenizer, microfluidizer or mixer, such as a roto-stator. See, e.g., Martin, et al., 1983, Physical Pharmacy: Physical Chemical Principles in the Pharmaceutical Sciences, Lea & Febiger Publishers, Philadelphia; Gennaro, et al., 1990, Remington's Pharmaceutical Sciences, 18^{th} edition, Mack Publishing Company, Easton, Pa. The preparation of lipids also can utilize other liposome forming techniques which may not be able to achieve the desired final average vesicle diameter or narrow range of vesicle diameters within a liposome preparation including, but not limited to, homogenization, microfluidization, sonication, high-shear mixing, or extrusion through metal frits or ceramic filters. See, e.g., New, 1990, Liposomes: A Practical Approach, Oxford University Press,

In another preferred embodiment, liposomes are formed under conditions of high encapsulation efficiency. A reverse evaporation phase method is preferred. Reverse-phase evaporation vesicles (REVs) formed by this method are characterized by (a) one or more bilayers, (b) an encapsulation efficiency typically between about 20-50% and (c) a broad spectrum of sizes between about 500 and up to 20,000 nm (20 microns). These and other liposome-preparation methods have been reviewed. See, Szoka, et al., 1980, Biochim. Biophys. Acta 601:559-71.

The preparation to be extruded also can comprise any substance that one wishes to encapsulate in or bind to the vesicle, micelle or liposome. In a preferred embodiment, the substance is cholesterol or other steroid or steroid derivative, solvent, buffer, acid, base, salt, metal, chelator, sugar, protein, nucleic acid or drug. See, e.g., Lasic, 1997, Liposomes in Gene Delivery, CRC Press LLC, Boca Raton 67-71. In a more preferred embodiment, the substance is cholesterol, polyethylene glycol, an alkylsulfate, ammonium bromide or albumin. In another more preferred embodiment, the substance is a drug. Liposomes can be used, for example, to alter the tissue distribution and uptake of drugs, in a therapeutically favorable way, and can increase the convenience of therapy, by allowing less frequent drug administration. See, e.g., Poznansky, et al., 1984, Pharmacol. Rev. 36:277-336. In a still more preferred embodiment the drug is an antihyperlipidemic agent. See, The Physicians' Desk Reference (54^{th} ed., 2000). In an even more preferred embodiment, the antihyperlipidemic agent is colestipol hydrochloride, ethyl 2-(p-chlorophenoxy)-2-methyl-proprionate, gemfibrozil, fenofibrate, cerivastatin sodium, fluvastatin sodium, atorvastatin calcium, lovastatin, pravastatin sodium, simvastatin or nicotinic acid. See id. In another more preferred embodiment, the drug is an antibiotic. In a still more preferred embodiment, the antibiotic is doxorubicin. See id. at 508. In another still more preferred embodiment, the antibiotic is amphotericin B. See id. at 1653. In another preferred embodiment, the anti-cancer drug is vincristine, mitoxantrone or other anti-cancer drugs. See, e.g. Bally, et al., 1990, Biochim. Biophys. Acta 1023: 133-9, Sugarman, et al., 1992, Crit. Rev. Oncol. Hematol. 12: 231-42, Kim, et al., 1993, Drugs 46: 618-38; Lim, 1997, J. Pharmacol. Exp. Ther. 281:566-73; Fielding, 1991, Clin. Pharmokinet. 21:155-64.

For liposomes intended for in vivo use, an aqueous buffer comprising a pharmaceutically acceptable carrier can be used. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, sodium phosphate, potassium chloride, calcium chloride, etc. A preferred embodiment uses an aqueous buffer that has approximately a physiological osmolality (i.e., 290 mOsm/kg). Examples of such buffers include 0.9% saline, 5% dextrose and 10% sucrose solutions. Many other pharmaceutically acceptable carriers may be employed. Generally, normal saline will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc.

The preparation to be extruded also may contain impurities or contaminants, although in a preferred embodiment these substances are removed from the aqueous solution either before, during or after the extrusion process.

These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration.

Uses of Liposomes

Vesicles, micelles and liposomes created using the methods and devices of the present invention can be used in any way that vesicles, micelles and liposomes created using conventional techniques can be used. In a preferred embodiment, liposomes created using the methods and devices of the present invention are used to deliver a drug or pharmaceutically active substance to a patient. See, e.g., U.S. Pat. Nos. 4,769,250; 4,906,477; 5,736,155; 6,060,080; Poznansky, et al., 1984, Pharmacol. Rev. 36:277-36; Lim, 1997, J. Pharmacol. Exp. Ther. 281:566-73; Kim, 1993, Drugs 46:618-38; Fielding, 1991, Clin. Pharmokinet. 21:155-64; Sugarman, et al., 1992, Crit. Rev. Oncol. Hematol. 12:231-42; Bally, et al., 1990, Biochim. Biophys. Acta 1023:133-39. In a more preferred embodiment, the liposome preferentially delivers the drug or pharmaceutically active substance to a tissue or cell type in the subject. In another preferred embodiment, the liposomes encapsulate a nucleic acid. See, Lasic, 1997, Liposomes in Gene Delivery, CRC Press LLC, Boca Raton 67-71. In an especially preferred embodiment the nucleic acid is an antisense nucleic acid used to inhibit expression of a gene. In another especially preferred embodiment the nucleic-acid containing liposome is used in a gene therapy protocol to treat, for example, a genetic disease (such as, e.g., cystic fibrosis, Gaucher's Diseases, sickle cell anemia, thalassemia, hemophilia or familial hypercholesterolemia), cancer (by, e.g., enhancing the immunogenicity of a tumor, enhancing the activity of immune cells, inserting a suicide gene into a tumor, inserting a tumor suppressor gene into a tumor, blocking the expression of a gene, protecting stem cells or inserting toxin-encoding genes under control of a tumor-specific promoter), an infectious disease (such as, e.g., acquired immune deficiency syndrome, hepatitis or herpes), a neurological disease (such as, e.g., Parkinson's disease, Alzheimer's disease or amyotrophic lateral sclerosis), a cardiovascular disease (such as, e.g., atherosclerosis, restenosis, thrombosis or heart ischemia), or another disease or condition (such as, e.g., arthritis, asthma, diabetes, osteoporosis, and infirmities associated with old age). See, Lasic, 1997, Liposomes in Gene Delivery, CRC Press LLC, Boca Raton 67-71 at pages 8-13.

In an especially preferred embodiment, the methods and devices of the present invention are used to make liposomes useful for treating atherosclerosis, as described in U.S. Pat. Nos. 5,746,223; 6,367,479; 6,079,416; 6,080,422; 5,736,157; 5,948,435; 5,858,400; 5,843,474; 6,312,719 and 6,139,871. The liposome can be bound to a protein or polypeptide to increase the rate of cholesterol transfer or the cholesterol-carrying capacity of the liposome. Binding of apolipoproteins to the liposomes is particularly useful. Apolipoprotein A₁, apolipoprotein A₂, and apolipoprotein E, or fragments, derivatives, agonists, analogues or peptide mimetics thereof, will generally be the most useful apolipoproteins to bind to the liposomes. See, e.g., U.S. Pat. Nos. 6,037,323; 6,004,925 and 6,046,166. These apolipoproteins promote transfer of cholesterol and cholesteryl esters to the liver for metabolism. Lecithin-cholesterol acyltransferase is also useful for metabolizing free cholesterol to cholesteryl esters. The liposomes may be bound to molecules of apolipoprotein A₁, apolipoprotein A₂, and lecithin-cholesterol acyltransferase, or fragments, derivatives, agonists, analogues or peptide mimetics thereof, singly or in any combination and molar ratio.

In a preferred embodiment, liposomes for treatment of a patient made according to the methods and devices of the present invention are present in a physiologically acceptable buffer, carrier or diluent. The concentration of liposomes in the buffer, carrier or diluent may vary. Generally, the concentration will be about 20-300 mg/ml, usually about 100-300 mg/ml, and most usually about 100-200 mg/ml. Persons of skill may vary these concentrations to optimize treatment with different liposomal components or of particular patients. For example, the concentration may be increased to lower the fluid load associated with treatment. This may be particularly desirable in patients having atherosclerosis-associated congestive heart failure or severe hypertension. Alternatively, liposomes composed of irritating lipids may be diluted to low concentrations to lessen inflammation at the site of administration.

### EXAMPLES

1. Extrusion of 20% POPC through 0.1 μm Polycarbonate Track-Etched Membranes at 600 psi

The following example demonstrates that a difficult lipid can clog or foul the extrusion membrane using conventional methods and devices, but that a flushing agent can be advantageously employed.

Two g of 1-palmitoyl-2-oleoyl-sn-glycero-3-phosphocholine (POPC) (Genzyme, Cambridge Mass., Cat. No. LP-04-031) was added to 8 ml of a phosphate buffered saline solution (PBS) (140 mM saline, 20 mM phosphate, pH ˜7.4) in a 50 ml conical tube and shaken vigorously by hand for about 5 min to form a homogenous 200 mg/ml suspension of POPC MLVs in PBS. A 10 ml LIPEX™ extruder (Northern Lipids, Vancouver, British Columbia, Canada) was fitted with a 2-stack of 0.1 μm polycarbonate track-etched (PCTE) NUCLEPORE™ membranes (Whatman, Ann Arbor, Mich.; Cat. No. 110605) according to the extruder manufacturer's instructions and flushed with PBS. The POPC-PBS MLV suspension was passed through the membrane stack at 600 psi. The total time required to extrude the 10 ml volume was 17 min 32 sec. A second pass through the same membrane stack was attempted but was aborted after 25 minutes. In that time, only about 2.5 ml of the suspension passed through the membrane stack, indicating that it had clogged or fouled. This was verified by adding ethanol to the system and continuing the extrusion. 100% ethanol was added to the extruder reservoir to bring the final concentration to 10% ethanol. Approximately 0.8 ml of 100% ethanol was added to the barrel, bringing its contents to about 10% ethanol, by swirling the barrel for 2 min. Application of 600 psi to the extruder allowed the extrusion to continue, indicating that the filters were clogged or fouled by the POPC MLVs.

2. Extrusion of 20% POPC through 2-Stacked PORETICS™ and NUCLEPORE™ PCTE Membranes at 400 and 800 psi

This example demonstrates that extrusion of a 20% POPC suspension proceeds with significantly less clogging or fouling with PORETICS™ PCTE membranes (Osmonics, Minnetonka, Minn.) than with NUCLEPORE™ PCTE membranes.

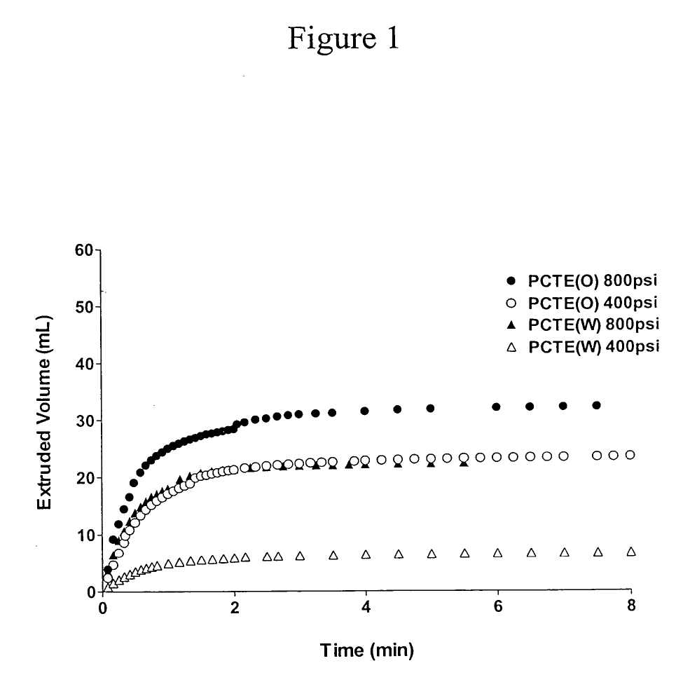

A 20% suspension of GENZYME™ POPC was prepared and extruded through a 10ml LIPEX™ extruder as described in Example 1. In separate trials, the extruder was configured with either a 2-stack of PORETICS™ or NUCLEPORE™ 0.1 μm avg. pore size PCTE membranes (Osmonics PORETICS™ Catalogue No. KOICPO2500 and Whatman NUCLEPORE™ Catalogue No. 110605, respectively). In separate trials, extrusions were conducted at 400 or 800 psi. As shown in FIG. 1, even at 400 psi PORETICS™ PCTE membranes clog or foul much less readily than NUCLEPORE™ PCTE membranes. This difference also is observed at 800 psi.

3. Extrusion of 20% POPC through Polyester Track-Etched (PETE) Membranes

This example demonstrates that a lipid can be passed through a hydrophilic extrusion membrane without fouling or clogging the membrane.

In separate trials, 10ml of a 200 mg/ml suspension of POPC MLVs in PBS (prepared as described in Example 1) was extruded through PORETICS™ polyester track-etched (PETE) membranes, Cat. No.s T04CP02500 (0.4 μm avg. pore diameter, 25 mm membrane diameter), T02CP047FX (0.2 μm avg. pore diameter, 47 mm membrane diameter (hand cut to a 25 mm diameter) and T01CP02500 (0.1 μm avg. pore diameter, 25 mm membrane diameter). The results are presented in Table 1. A fresh batch of POPC MLVs was made for each trial. The extruder was flushed with saline between each trial. The average particle size of the extruded suspension was determined by QELS using a 380 ZLS particle sizer (Nicomp, Santa Barbara, Calif.) according to the manufacturer's instructions.

TABLE 1

| | | Avg. Pore | No. of | | | |
|---|---|---|---|---|---|---|
| | | Diameter | Membranes | Pressure | Time | Avg. Particle |
| Trial No. | Pass No. | (μm) | in Stack | (psi) | (min:sec) | Size (nm) |
| 1 | 1 | 0.4 | 1 | 100 | 1:03 | n.d. |
| 2 | 1 | 0.2 | 2 | 100 | 3:20 | n.d. |
| | 2 | ″ | ″ | ″ | 1:33 | 301.7 ± 121 |
| 3 | 1 | 0.1 | 2 | 200 | 0:48 | n.d. |
| | 2 | ″ | ″ | ″ | 0:34 | n.d. |
| | 3 | ″ | ″ | ″ | 0:30 | 184 ± 56 |
| | 4 | ″ | ″ | ″ | 0:29 | 175 ± 44 |
| 4 | 1 | 0.1 | 4 | 200 | 1:22 | 169 ± 37 |
| 5 | 1 | 0.1 | 4 | 400 | 0:23 | 158 ± 39.5 |
| 6 | 1 | 01. | 4 | 800 | 0:06 | 136 ± 42 |

These results demonstrate that PETE membranes do not clog or foul under conditions where PCTE membranes clog or foul. Furthermore, these results demonstrate that the particle size resulting from extrusion of a lipid can be reduced by increasing the extrusion pressure.

4. Extrusion of 20% EPC through 5-Stacked PETE Membrane at High and Low Pressure

This example demonstrates that a relatively easy lipid can be extruded through a hydrophilic membrane.

A 20% solution of EPC was made by bringing 6 g of LIPOID EPC® (Lipoid, Ludwigshafen, Germany), a phosphatidylcholine from egg yolk, to a total of 30 ml in saline solution (Abbot, Abbott Park, Ill.) in a 50 ml conical flask. The flask was shaken by hand for about 5 min until visually homogeneous. A 10 ml LIPEX™ extruder was set up with a 5 stack of 0.1 μm avg. pore diameter, 25 mm membrane diameter PORETICS™ PETE membranes (Cat. No. T01CP02500). In two separate trials, 10 ml of the EPC suspension was passed 10 times through the filter at either 400 or 800 psi. The extruder was cleaned between trials. The results of these trials are shown in Table 2.

TABLE 2

| | | | | Avg. Particle |
|---|---|---|---|---|
| Trial | Pass No. | Pressure (psi) | Time (min:sec) | Size (nm) |
| 1 | 1 | 400 | 1:52 | n.d. |
| | 2 | ″ | 0:32 | n.d. |
| | 3 | ″ | 0:27 | n.d. |
| | 4 | ″ | 0:25 | n.d. |
| | 5 | ″ | 0:24 | 181.0 ± 52.9 |
| | 6 | ″ | 0:25 | n.d. |
| | 7 | ″ | 0:27 | n.d. |
| | 8 | ″ | 0:28 | n.d. |
| | 9 | ″ | 0:30 | n.d. |
| | 10 | ″ | 0:33 | 153.6 ± 45.2 |
| 2 | 1 | 800 | 0:42 | n.d. |
| | 2 | ″ | 0:17 | n.d. |
| | 3 | ″ | 0:16 | n.d. |
| | 4 | ″ | 0:15 | n.d. |
| | 5 | ″ | 0:18 | 138.6 ± 35.6 |
| | 6 | ″ | 0:18 | n.d. |
| | 7 | ″ | 0:19 | n.d. |
| | 8 | ″ | 0:19 | n.d. |
| | 9 | ″ | 0:22 | n.d. |
| | 10 | ″ | 0:25 | 114.4 ± 35.8 |

Thus, high pressure extrusion of EPC through PETE membranes increases flow rates and reduces particle size.

5. Extrusion of 20% POPC through a Single PCTE or PETE Membrane at 800 psi

This example demonstrates that extrusion through a PETE membrane produces a greater flow rate than extrusion through a PCTE membrane.

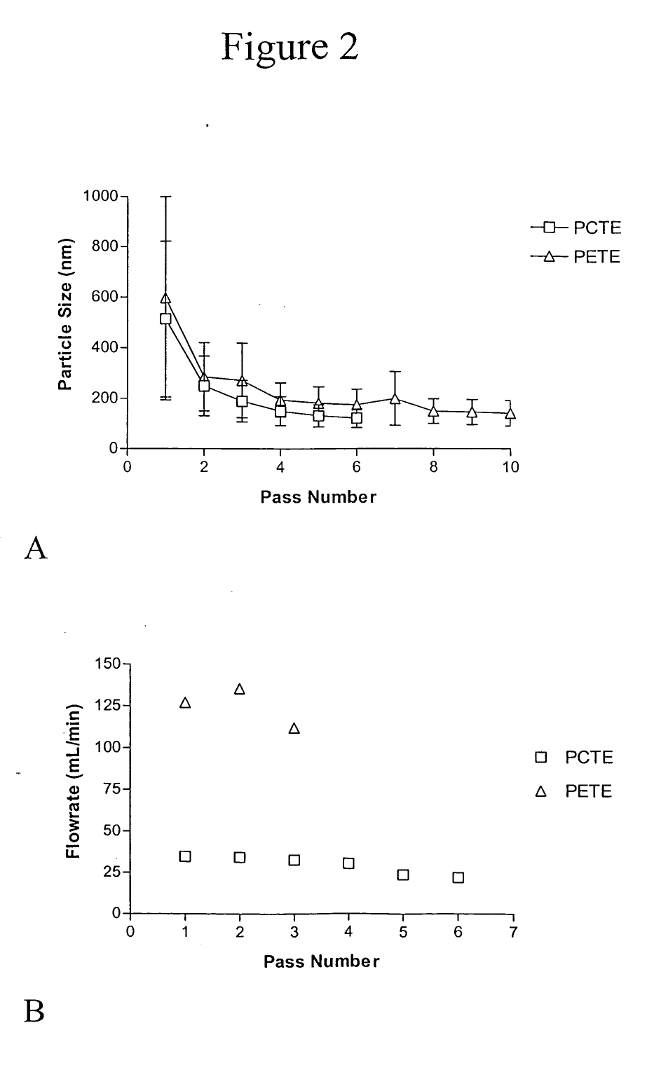

A 20% suspension of GENZYME™ POPC was prepared and extruded through a 10 ml LIPEX™ extruder as described in Example 1. In separate trials, the extruder was configured with either a single PORETICS™ 0.1 μm avg. pore size PETE membrane or a single PORETICS™ 0.1 μm avg. pore size PCTE membrane (Osmonics Catalogue No.s T01CP02500 and K01CP02500, respectively). As shown in FIG. 2, under these conditions, the PCTE membrane produced smaller particles with fewer passes than the PETE membrane (FIG. 2A). However, extrusion through the PETE membrane occurred with a flow rate that was about 3 times greater than the flow rates achieved with the PCTE membrane (FIG. 2B).

6. Extrusion of 20% POPC through a 5-Stack PETE Membrane at 600 psi

This example demonstrates that a suspension of a difficult lipid can be efficiently converted into a suspension of SUVs by extrusion through a hydrophilic membrane at moderately high pressure.

A 20% GENZYME™ POPC suspension was made as described in Example 1. A 10 ml LIPEX™ extruder was set up with a 5-stack of PORETICS™ 0.1 μm avg. pore size PETE membranes (Cat. No. T01CP02500). The POPC suspension was extruded through the membranes in five passes at 600 psi. The results are shown in Table 3.

TABLE 3

| | | | Avg. Particle |
|---|---|---|---|
| Pass No. | Pressure (psi) | Time (min:sec) | Size (nm) |
| 1 | 600 | 2:37 | n.d. |
| 2 | ″ | 1:19 | n.d. |
| 3 | ″ | 1:02 | n.d. |
| 4 | ″ | 0:59 | n.d. |
| 5 | ″ | 0:55 | 118.1 ± 36.2 |

7. Extrusion of POPC through 2-stack PCTE and PETE Membranes at 400 and 800 psi

This example demonstrates that PETE membranes clog or foul much less readily than angled pore PCTE membranes at high pressure.

In some of our earlier experiments, a portion of the extruded lipid suspension was lost post-extrusion due to the excess nitrogen gas escaping at high velocities out of the exit collection tubing at the end of extrusions carried out at high pressure. This escaping gas frequently caused the exit tubing to be blown off of the extruder base and also caused some of the product solution to be splashed out of the collection container. To remedy this problem, we set up a guard tubing to prevent the exit collection tube from being blown off of the base of the extruder. This guard tubing was essentially a larger diameter piece of tubing that the thinner exit collection tube was threaded through. The guard tubing provided extra friction to the exit collection tube from the base plate. For additional control of the extruding solution, a ring stand was set up to act as a guide, forcing the tubing to remain in the correct orientation, with the extruded suspension being adequately collected by our collection vessel. These two additions to the apparatus added enough additional control over the exit collection tubing to prevent most product loss.

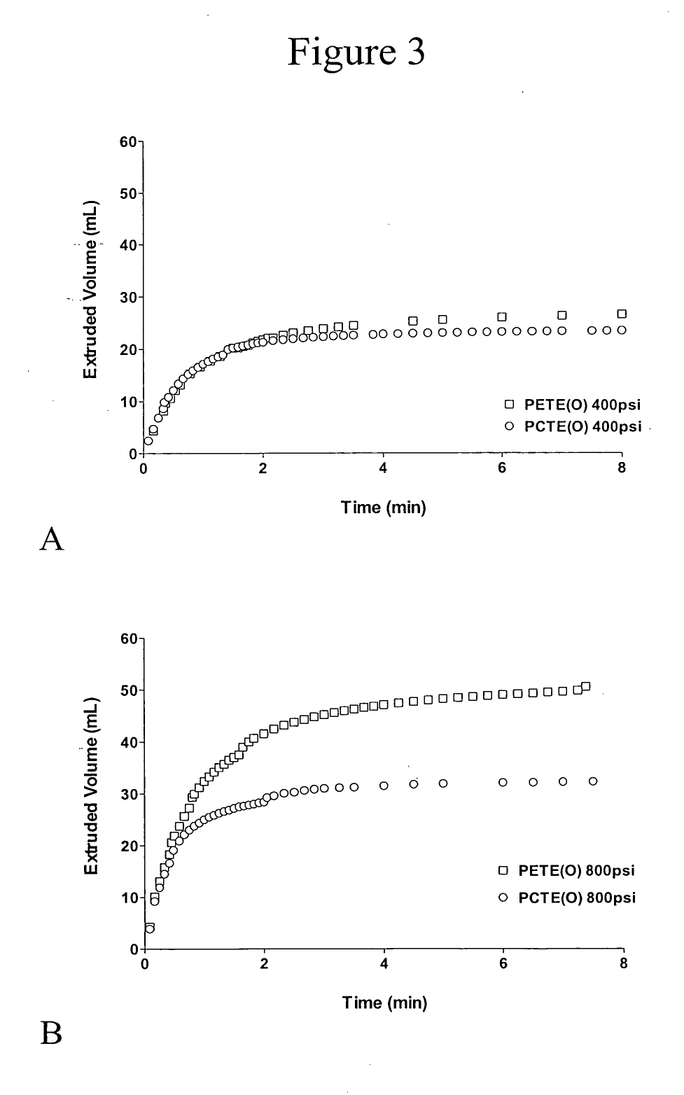

A 20% GENZYME™ POPC suspension was made as described in Example 1. In separate trials, a 10 ml LIPEX™ extruder was set up with a 2-stack of PORETICS™ 0.1 μm avg. pore size PETE (Cat. No. T01CPO2500) or PCTE (Cat. No. K01CP02500) membranes. For each of these setups, the POPC suspension was extruded through the membranes at either 400 or 800 psi, and the volume of extruded suspension that could be processed in one pass measured as a function of time. The results are presented in FIG. 3. FIG. 3A shows that at 400 psi there is not a significant difference between the PETE and the PCTE membranes. FIG. 3B shows that at high pressure the PETE membrane configuration can process a significantly larger volume than the PCTE membrane configuration before the membrane becomes clogged or fouled.

8. Extrusion of 20% POPC through 1-, 2-, 5- and 10-Stack PETE Membranes

The following example demonstrates the effects of membrane number on the efficiency of producing POPC LUVs.

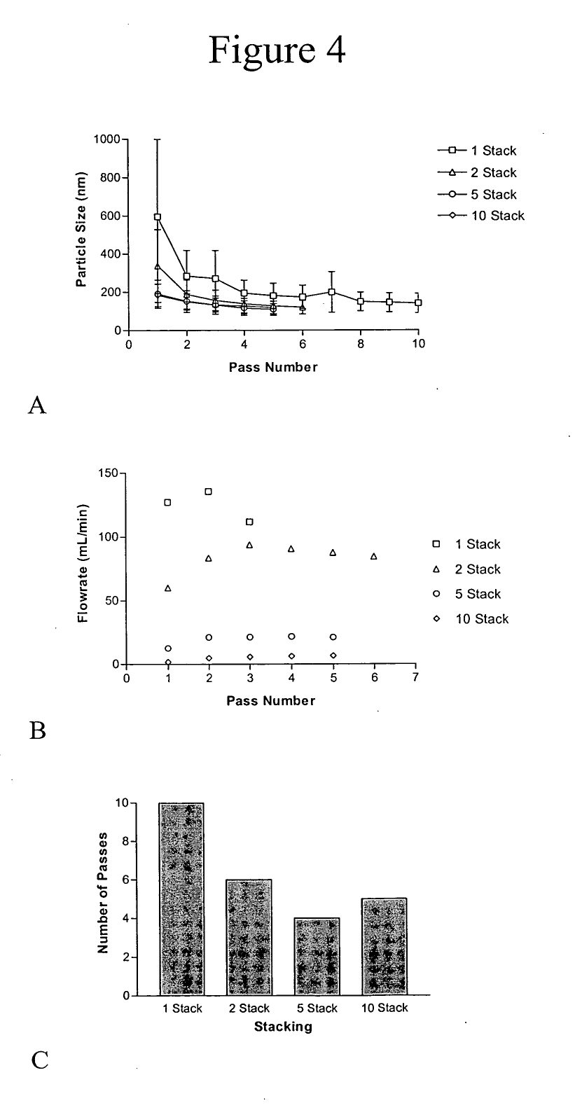

A 20% suspension of GENZYMET POPC was prepared and extruded through a 10ml LIPEX™ extruder as described in Example 1. In separate trials, the extruder was configured with either 1, 2, 5 or 10 stacked PORETICS™ 0.1 μm avg. pore size PETE membranes (Osmonics Poretics Catalogue No. T01CP02500). All extrusions were conducted at a pressure of 800 psi. The results are shown in FIG. 4. FIG. 4A shows the relationship between pass number and the average particle diameter of the LUVs produced. There is generally an inverse correlation between the number of membranes in the stack and the number of passes required to produce LUVs of a desired average diameter. FIG. 4B shows the relationship between pass number and flow rate. For any given pass number, the flow rate is inversely proportional to the number of filters in the stack. As shown in FIG. 4C, the number of passes required to produce LUVs of an average diameter of 120 nm is slightly lower for the 5-stack (4 passes) than it is for the 10-stack (5 passes).

9. Extrusion of 20% POPC through a 5-Stack of PETE Membranes at 400, 600 and 800 psi

This example demonstrates the effects of pressure on the efficiency of producing POPC LUVs.

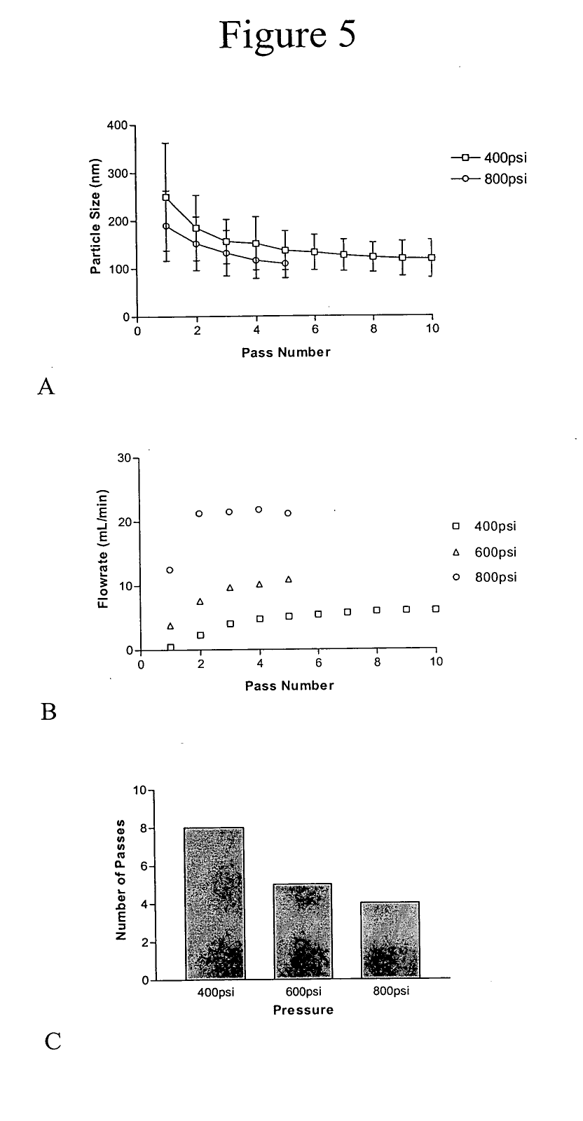

A 20% suspension of GENZYMET POPC was prepared and extruded through a 10 ml LIPEX™ extruder as described in Example 1. The extruder was configured with a 5-stack of PORETICS™ 0.1 μm avg. pore size PETE membranes (Osmonics PORETICS™ Catalogue No. T01CP02500). In separate trials, the extrusion was performed at either 400, 600 or 800 psi. As shown in FIG. 5A, particles of smaller size were achieved after fewer passes using 600 or 800 psi as compared to 400 psi. FIG. 5B shows that the flow rate for a given pass was about twice as great at 800 psi than at 600 psi, and the flow rate at 600 psi was about twice as great as the flow rate at 400 psi. FIG. 5C shows that to produce LUVs with an average diameter of about 120 nm, 8 passes are needed at 400 psi, 5 passes at 600 psi and 4 passes at 800 psi.

10. Extrusion of GENZYME™ POPC through 2-Stack PCTE and PETE PORETICS™ and PCTE NUCLEPORE™ Membranes at 400 to 1.500 psi

This example demonstrates that increasing the pressure of extrusion through a PETE membrane causes an unexpectedly large decrease in fouling or clogging of the membrane, and thus an unexpectedly large increase in the lipid processing capacity of the membrane, as compared to extrusion through a PCTE membrane. This example further demonstrates that there is no apparent upper limit to this effect.

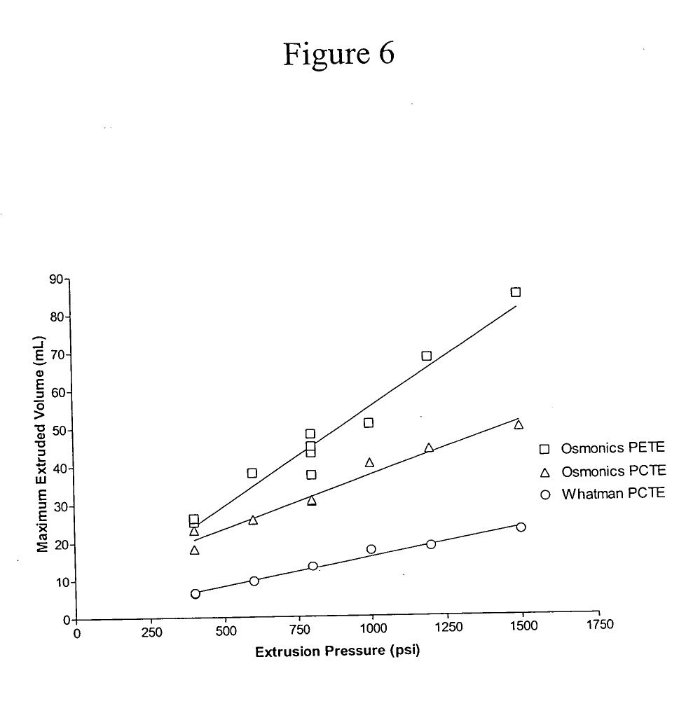

A 20% POPC suspension was made as described above. In separate trials, a 10 mL LIPEX™ extruder was set up with either 2-stacked PORETICS™ PETE or PCTE or NUCLEPORE™ PCTE 0.1 μm pore size membranes (Osmonics PORETIC™ Catalogue No.s T01CP02500, K01CP02500, and Whatman Nuclepore Catalogue No. 110605, respectively). For each of these setups, the POPC suspension was extruded through the membranes at pressures ranging from 400 psi to 1,500 psi. The weight of the POPC suspension extruded was measured as a function of time, and the maximum amount of suspension each membrane was able to pass at the given pressure was calculated. These calculations were plotted against the extrusion pressure as shown in FIG. 6. For each membrane type used, the maximum amount of suspension processed before the membrane became completely clogged increased linearly with increasing extrusion pressure. There is no apparent upper limit to the linearity of this increase. The slope of each membrane's plot is a measure of the degree of improvement for the membrane as the pressure is increased. The slopes for the plots of results for PORETICS™ PETE, PORETICS™ PCTE and NUCLEPORE™ PCTE membranes are 0.051, 0.028 and 0.015, respectively.

11. Extrusion of 20% POPC through a Step-Down of 2-Stacked PETE Membranes at Pressures Up to 5.000 and 8.000 psi.

This example demonstrates that using higher extrusion pressures reduces particle size more rapidly and therefore the desired particle size is achieved in fewer extrusion passes. The example is similar to that described in Example 9 (FIG. 5C) except in this example a step-down extrusion processing method was used in combination with higher extrusion pressures and the material was passed through double-stacks of membranes with decreasing pore size diameters. Because of higher extrusion pressures, fewer extrusion passes are required to reach the desired particle size and hence overall processing time is significantly reduced.

In separate trials, 20% POPC suspensions were made by hydrating POPC in phosphate buffered saline solution. The resulting solutions were then passed as discrete passes through extrusion membranes at extrusion pressures up to either 5,000 or 8,000 psi. The first extrusion pass was through a 2-stacked PORETICS™ PETE 0.4 μm membranes, the second pass was through a 2-stacked PORETICS™ PETE 0.2 μm membranes, and the remaining passes were through 2-stacked PORETICS™ PETE 0.1 μm membranes. Particle size measurements were performed after each extrusion pass. The data in Table 4 shows that the number of passes required to achieve an average particle size diameter of less than 140nm is reduced due to higher pressures.

TABLE 4

| | | | Number of Extrusion passes |
|---|---|---|---|
| | | | required to reach average |
| Extrusion | | | particle size diameter of less |
| Pressure (psi) | Production Scale | | than 140 nm |
| 5,000 | ˜350 | mL | 5 |
| 5,000 | ˜500 | mL | 5 |
| 8,000 | ˜650 | mL | 4 |
| 8,000 | ˜55 | L | 4 |
| 8,000 | ˜72 | L | 3 |

12. Extrusion of 20% POPC through 2-Stacked Whatman ANOPORE™ Membranes at Pressures up to 1.500 psi.

This example demonstrates that using higher extrusion pressures increases the extrusion volume and reduces clogging or fouling. In this example a 20% POPC suspension was made and extruded through a 2-stack of 0.1 μm Whatman ANOPORE™ aluminum oxide inorganic membranes at varying pressures. A greater volume of material was able to flow through the membranes at higher pressure.

TABLE 5

| Extrusion | Maximum Extruded Volume |
|---|---|
| Pressure (psi) | of 20% POPC (mL) |
| 400 | 2.06 |
| 800 | 9.87 |
| 1,200 | 22.09 |
| 1,500 | 27.27 |

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below.

All references cited herein are incorporated by reference in their entireties.

1. A method of producing a suspension of vesicles comprising extruding a mixture comprising a lipid through a hydrophilic screen membrane at high pressure.

2. The method of claim 1 wherein said suspension of vesicles is a suspension of liposomes.

3. The method of claim 1 wherein said mixture comprises a suspension of multilaminate vesicles.

4. The method of claim 1 wherein said mixture is an emulsion.

5. The method of claim 1 wherein said mixture comprises a plurality of lipids.

6. The method of claim 1 wherein said hydrophilic screen membrane has a water contact angle of about 70 degrees or less.

7. The method of claim 6 wherein said screen membrane has a water contact angle of about 50 degrees or less.

8. The method of claim 7 wherein said screen membrane has a water contact angle of about 40 degrees or less.

9. The method of claim 1 wherein said hydrophilic screen membrane comprises at least one material selected from the group consisting of polyester, aluminum oxide, cellulose acetate, cellulose mixed ester, glass, polyethersulfone, polyvinyl pyrolidine and polysulfone.

10. The method of claim 1 wherein said hydrophilic screen membrane is a polyester membrane.

11. The method of claim 1 wherein said hydrophilic screen membrane is a track-etched membrane.

12. The method of claim 1 wherein said hydrophilic screen membrane comprises a coating.

13. The method of claim 12 wherein said coating is a hydrophilic coating.

14. The method of claim 12 wherein said coating is a hydrophobic coating.

15. The method of claim 1 wherein said vesicles have an average diameter of between about 50 nm and 400 nm.

16. The method of claim 1 wherein said vesicles have an average diameter of between about 50 nm and 150 nm.

17. The method of claim 1 wherein said vesicles have an average diameter of between about 100 nm and 150 nm.

18. The method of claim 1 wherein said vesicles have an average diameter in the range of about 169±37 nm.

19. The method of claim 1 wherein said vesicles have an average diameter in the range of about 158±39.5 nm.

20. The method of claim 1 wherein said vesicles have an average diameter in the range of about 136±42 nm.

21. The method of claim 1 wherein said vesicles have an average diameter in the range of about 153.6±45.2 nm.

22. The method of claim 1 wherein said vesicles have an average diameter in the range of about 138.6±35.6 nm.

23. The method of claim 1 wherein said vesicles have an average diameter in the range of about 114.4±35.8 nm.

24. The method of claim 1 wherein said vesicles have an average diameter in the range of about 118.1±36.2 nm.

25. The method of claim 1 wherein said lipid has a transition temperature at or below room temperature.

26. The method of claim 1 wherein said lipid has a transition temperature above room temperature.

27. The method of claim 1 wherein said lipid comprises a rigid acyl chain.

28. The method of claim 27 wherein said rigid acyl chain is a mono-unsaturated acyl chain.

29. The method of claim 1 wherein the mixture comprises impurities or contaminants.

30. The method of claim 1 wherein the lipid is a drug-associated lipid.

31. The method of claim 1 wherein the lipid is a charged lipid.

32. The method of claim 1 wherein the lipid is associated with a protein.

33. The method of claim 1 wherein said lipid is selected from the group consisting of 1-palmitoyl-2-oleoyl-sn-glycero-3-phosphocholine, dipalmitoylphosphatidylcholine, dipalmitoylphosphatidylglycerol di-stearoyl-phosphatidylethanolamine, egg yolk phosphatidylcholine, di-oleoyl-phosphatidylcholine, di-lauroylphosphatidylcholine, di-lauroylphosphatidylglycerol, oleoyl-palmitoylphosphatidylcholine, glycolipid-linked phospholipids, phosphatidylcholine, phosphatidylglycerol, lecithin, β, γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, di-oleoyl-phosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolarnine, di-myrstoyl-phosphatidylserine and di-oleyl-phosphatidylcholine.

34. The method of claim 33 wherein the lipid is phosphatidylcholine or sphingomyelin.

35. The method of claim 1 wherein said hydrophilic screen membrane has an average pore diameter of about 0.4 μm or less.

36. The method of claim 35 wherein said hydrophilic screen membrane has an average pore diameter of about 0.2 μm or less.

37. The method of claim 36 wherein said hydrophilic screen membrane has an average pore diameter of about 0.1 μm or less.

38. The method of claim 1 wherein said extrusion is performed at a pressure of about 400 psi or greater.

39. The method of claim 38 wherein said extrusion is performed at a pressure of about 800 psi or greater.

40. The method of claim 39 wherein said extrusion is performed at a pressure of about 1,500 psi or greater.

41. The method of claim 40 wherein said extrusion is performed at a pressure of about 5,000 psi or greater.

42. The method of claim 41 wherein said extrusion is performed at a pressure of about 8,000 psi or greater.

43. The method of claim 42 wherein said aqueous suspension of lipids is extruded through a plurality of stacked membranes.

44. The method of claim 43 wherein each stacked membrane has the same average pore diameter.

45. The method of claim 44 wherein at least one stacked membrane has an average pore diameter different from the average pore diameter of at least one other stacked membrane.

46. The method of claim 45 wherein said stacked membranes are arranged so that said mixture is extruded through membranes of progressively smaller average pore size.

47. The method of claim 1 wherein said extrusion is conducted at a controlled temperature.

48. The method of claim 47 wherein said controlled temperature is approximately constant temperature.

49. The method of claim 48 wherein said approximately constant temperature is about room temperature.

50. The method of claim 49 wherein said approximately constant temperature is between about 20° C. to about 30° C.

51. The method of claim 50 wherein said approximately constant temperature is about 25° C.

52. The method of claim 1 wherein said mixture is extruded through said hydrophilic membrane at a flux rate of between about 0.0001 and about 40 mL/min/mm².

53. The method of claim 1 wherein said vesicles comprise a pharmaceutically active substance.

54. The method of claim 1 wherein said extrusion comprises multiple passes.

55. The method of claim 54 wherein said extrusion comprises a step-down extrusion.

56. The method of claim 1 wherein said mixture is extruded through said hydrophilic screen membrane alternately in the forward and reverse directions.

57. The method of claim 1 wherein said hydrophilic screen membrane has a pore density greater than about 8×10⁵ pores/cm².

58. The method of claim 1 wherein said hydrophilic screen membrane has a thickness of between about 3 and about 50 μm.

59. A device for extruding an aqueous suspension of lipids at high pressure comprising a hydrophilic screen membrane and means for entry and exit of liquid under high pressure.

60. The method of claim 1 wherein said hydrophilic screen membrane is rinsed with a flushing agent prior to said extrusion.

61. The method of claim 60 wherein said flushing agent removes clogged or fouled material from said membrane's pores.

62. The method of claim 60 wherein said flushing agent prevents clogged or fouled material from said membrane's pores.

63. The method of claim 61 or 62 wherein said flushing agent comprises ethanol.

64. A method of producing liposomes comprising extruding a mixture comprising a lipid through a hydrophilic membrane at pressures greater than about 8,000 psi.

65. The method of claim 64, wherein said vesicles have an average diameter of between about 50 nm and 400 nm.

66. The method of claim 64 herein the liposomes have an average diameter of between about 50 nm and 150 nm.

67. The method of claim 64 wherein the liposomes have an average diameter of between about 100 nm and 150 nm.

68. The method of claim 64 wherein said vesicles have an average diameter in the range of about 169±37 nm.

69. The method of claim 64 wherein said vesicles have an average diameter in the range of about 158±39.5 nm.

70. The method of claim 64 wherein said vesicles have an average diameter in the range of about 136±42 nm.

71. The method of claim 64 wherein said vesicles have an average diameter in the range of about 153.6±45.2 nm.

72. The method of claim 64 wherein said vesicles have an average diameter in the range of about 138.6±35.6 nm.

73. The method of claim 64 wherein said vesicles have an average diameter in the range of about 114.4±35.8 nm.

74. The method of claim 64 wherein said vesicles have an average diameter in the range of about 118.1±36.2 nm.
